(19) United States
(12) Patent Application Publication
(10) Pub. No.: US 2015/0305772 A1
(43) Pub. Date: Oct. 29, 2015

(54) MORCELLATION CONTAINMENT SYSTEM

(71) Applicant: Womens Health and Wellness, PLLC., Knoxville, TN (US)

(72) Inventor: Lowell L. McCauley, Knoxville, TN (US)

(21) Appl. No.: 14/697,015

(22) Filed: Apr. 27, 2015

Related U.S. Application Data
(60) Provisional application No. 61/984,235, filed on Apr. 25, 2014.

(51) Int. Cl. A61B 17/34; A61B 17/32; A61B 17/00

(52) U.S. Cl. CPC A61B 17/3431; A61B 17/3423; A61B 17/00234; A61B 17/32002; A61B 2017/00287; A61B 2017/320024

(57) ABSTRACT

A morcellation containment system including an expandable morcellation containment bag having a plurality of ports defined thereon for collecting and isolating a diseased organ or mass from healthy tissue for morcellation. Each port has an associated trocar seal that is sealable via a double squeegee valve.

### CROSS-REFERENCE TO RELATED APPLICATIONS

This application claims the benefit of U.S. Provisional Application No. 61/984,235, filed Apr. 25, 2014, which is incorporated herein in its entirety by reference.

### STATEMENT REGARDING FEDERALLY SPONSORED RESEARCH OR DEVELOPMENT

Not Applicable

### BACKGROUND OF THE INVENTION

1. Field of Invention

This invention pertains to a surgical containment system, and more particularly, it relates to a morcellation containment system including a morcellation containment bag useful in containing unwanted spillage and preventing contamination of healthy tissue, i.e. seeding of healthy tissue with diseased cells, during a laparoscopic surgery involving a power morcellation procedure.

2. Description of the Related Art

In the field of minimally invasive surgery, removal of diseased organs, or tumors and/or organs is commonly performed laparoscopically. It is known that in order to remove a diseased organ or tumor through the type of small incisions that are commonly used for laparoscopic surgery, the diseased organ or tumor must be cut into significantly smaller pieces. This is commonly done with an electrosurgical instrument known as a morcellator. While there are various types of power morcellators, a known risk of using a power morcellator, especially with a cancerous organ or tumor, is the risk of seeding healthy tissue with cancerous cells if small morcellated particulate matter is allowed to migrate within the abdominal, or cervical, cavity. Published International Patent Application No. PCT/US2012/065859, (“the '859 application”), having a priority date of 18 Nov. 2011, describes the use of a sealable retrieval bag for receiving a diseased organ prior to morcellation. However, even when using a sealable bag such as described in the '859 application, it is known that there is still a risk of cross-contamination as the trocars, laparoscope, morcellator, etc. are removed from the ports in the bag of the '859 application through the body cavity and removed from the incisions in the body wall. Further, morcellating tissue in known prior art tissue bags that do not provide access for a laparoscope, can also obscure the surgeon's view of the tissue being morcellated. What is missing from the art is a morcellator surgical bag that isolates a diseased organ or tumor from the abdominal cavity thereby substantially preventing the risk of seeding healthy tissue with cancerous tissue as surgical instruments are removed from the surgical bag. What is further missing from the known art is a morcellator bag that isolates the interior of the bag from healthy tissue without obscuring the surgeon's view of the tissue being morcellated.

Accordingly, it is an object of the present invention to provide a morcellation containment system which is sealed to the inner surface of the abdominal wall. It is a further object to provide a morcellation containment system which is inflatable so as to retract or mobilize vital organs from the surgical field. Still another object of the present invention is to provide a morcellation containment system which can be accessed by a standard laparoscope thereby providing direct visualization of the morcellation process. Yet another object of the present invention is to provide a morcellation containment system that provides for continuous gas flow thereby allowing use of any type of morcellator, including without limitation, an electrosurgical morcellator. A further object of the present invention is to provide a morcellation containment system which specifically addresses the needs of Trans-Cervical Morcellation and to specifically address and allow for trans-cervical or trans-vaginal morcellation.

### BRIEF SUMMARY OF THE INVENTION

Described herein is a novel morcellation containment system utilizing an expandable surgical bag, created from FDA approved materials, referred to herein as a morcellation containment bag, for collecting tissue, such as a diseased organ, through an incision in the body during endoscopic or laparoscopic surgery. The morcellation containment bag includes a plurality of trocar access ports defined therein by, but not limited to, double-squeegee type valved seals, or other types of gas sealing access ports, that are adapted to be brought into substantial alignment with the incisions in the abdominal wall through which the trocars, morcellator, laparoscope, etc. are inserted. Each of the trocar access ports include at least a pair of suture loops which the surgeon can grasp and pull upward, as described in greater detail below, sealing the upper portion of the trocar access port with interior lining of the abdominal wall. By sealing the trocar access ports of the morcellation containment bag against the interior lining of the abdominal wall, such that the ports defined in the morcellation containment bag substantially align with the laparoscopic or endoscopic incisions in the abdominal wall, a substantially sealed environment is defined within the interior volume of the morcellation containment bag. Further, a prophylactic type sleeve made from FDA approved materials may also be utilized which extends externally through the incision to further protect tissues from contamination.

In order to further isolate the interior volume of the morcellation containment bag from the abdominal cavity, the morcellation containment system of the present invention further may include, in an exemplary embodiment, trocars having selectively removable sleeves in order to prevent a contaminated sleeve from being exposed to healthy tissue, such as the healthy tissue at the incision site. It will be appreciated by those skilled in the art that trocars with removable sleeves are not, however, required. In use, the morcellation containment bag is inserted into the abdominal cavity through one of the incisions. It will be appreciated that different types of surgery require different insertion points, it being understood that the morcellation containment bag could also be inserted into the body via a cervical or vaginal route. Upon insertion, the diseased tissue or organ can be freed from surrounding healthy tissue and received within the interior volume of the morcellation containment bag through the opening in the bag.

The drawstring at the opening of the morcellation containment bag is grasped and brought through an opening defined by an incision in the abdominal wall. The morcellator is then inserted through the opening in the morcellation containment bag, and the morcellation containment bag opening is then sealed against the body of the morcellator prior to beginning morcellation. With all of the laparoscopic ports of the trocar access ports sealed up against inner abdominal wall by means of the suture loops, the interior of the morcellation containment bag is isolated from the abdominal cavity. In an exemplary embodiment, the morcellation containment bag can be inflated with a gas, commonly CO2 gas, while the diseased tissue or organ is contained within the morcellation containment bag; and a flow of gas can be maintained through the morcellation containment bag preventing vaporization hazards.

At the end of the procedure, the instruments can be removed from the trocars without coming into contact with the inner belly wall. Finally, the upper body of each trocar is detached from the trocar sleeve. The sleeves are then pushed, or alternatively pulled, back into the interior of the morcellation containment bag, and can, subsequently, be removed from the morcellation containment bag through the exteriorized lateral opening. The morcellation containment bag can then be removed with all residual blood, debris, and tissue splatter contained within. Alternatively, trocars with non-detachable sleeves can be used; and once they are removed, the suture loops can be pushed, pulled, or withdrawn into the morcellation bag cavity within the prophylactic type sheath, if this embodiment is being used, in order to further try to isolate healthy tissue and minimize external contamination from within.

### BRIEF DESCRIPTION OF THE DRAWINGS

The above-mentioned and additional features of the invention will become more clearly understood from the following detailed description of the invention read together with the drawings in which:

FIG. 1 is a perspective view of one embodiment of a morcellation containment bag for the morcellation containment system of the present invention constructed in accordance with the present invention;

FIG. 2 is schematic view, in partial cross-section, of the morcellation containment bag of FIG. 1 positioned within the abdominal cavity of a patient;

FIG. 3 is a schematic view, in partial cross-section, of the morcellation containment bag of FIG. 1 illustrating the flow of gas to expand the morcellation containment bag and prevent vaporization hazards; it being appreciated that FIG. 3 and the remaining Figures are not drawn to strict scale for clarity of view;

FIGS. 4A-4E are schematic views, in partial cross section, showing the various steps for deploying and securing the morcellation containment bag of the morcellation containment system of the present invention.

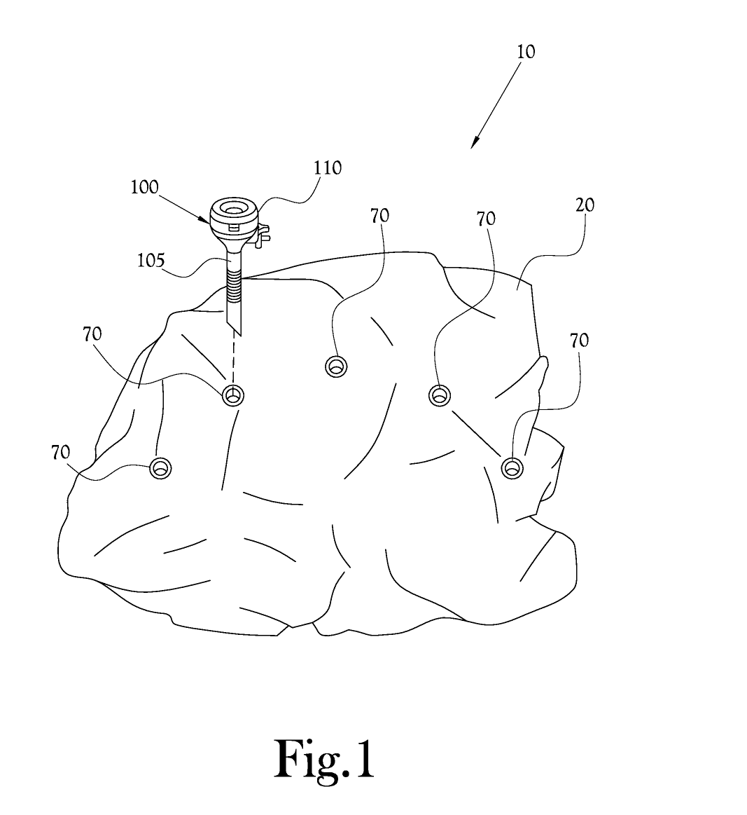

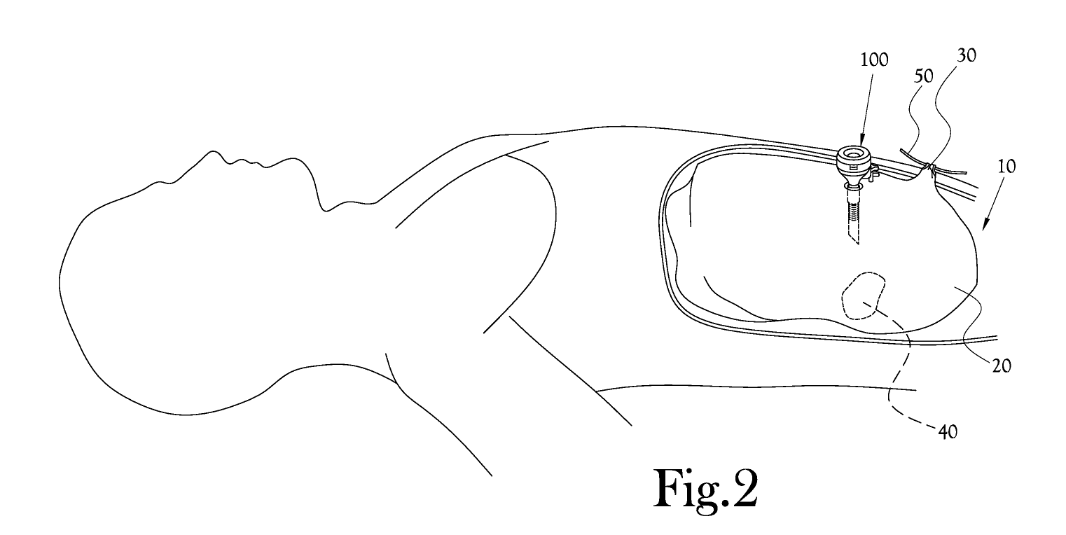

### DETAILED DESCRIPTION OF THE INVENTION

It will be recognized by those skilled in the art that there are a number of preliminary, or primary, steps or procedures that must be performed by the surgical team in order to prepare a patient for laparoscopic surgery. In the following detailed description of the components and the use of the morcellation containment system of the present invention, it is assumed that these preliminary, or primary, procedures have been completed and that there is security of hemostasis in the surgical field.

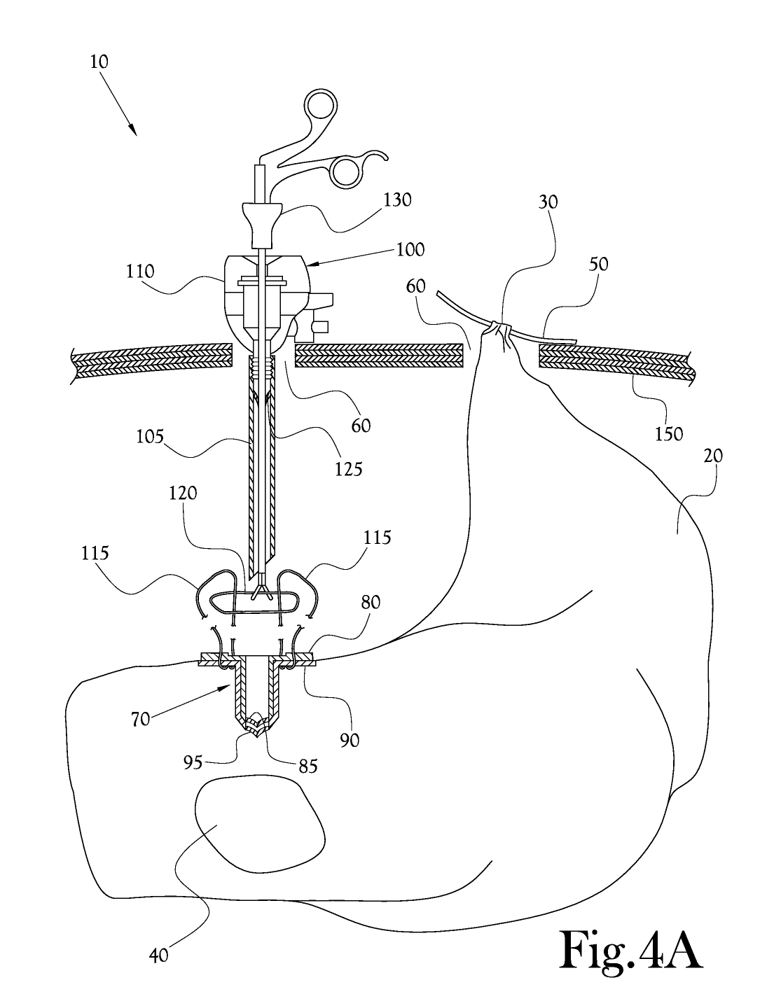

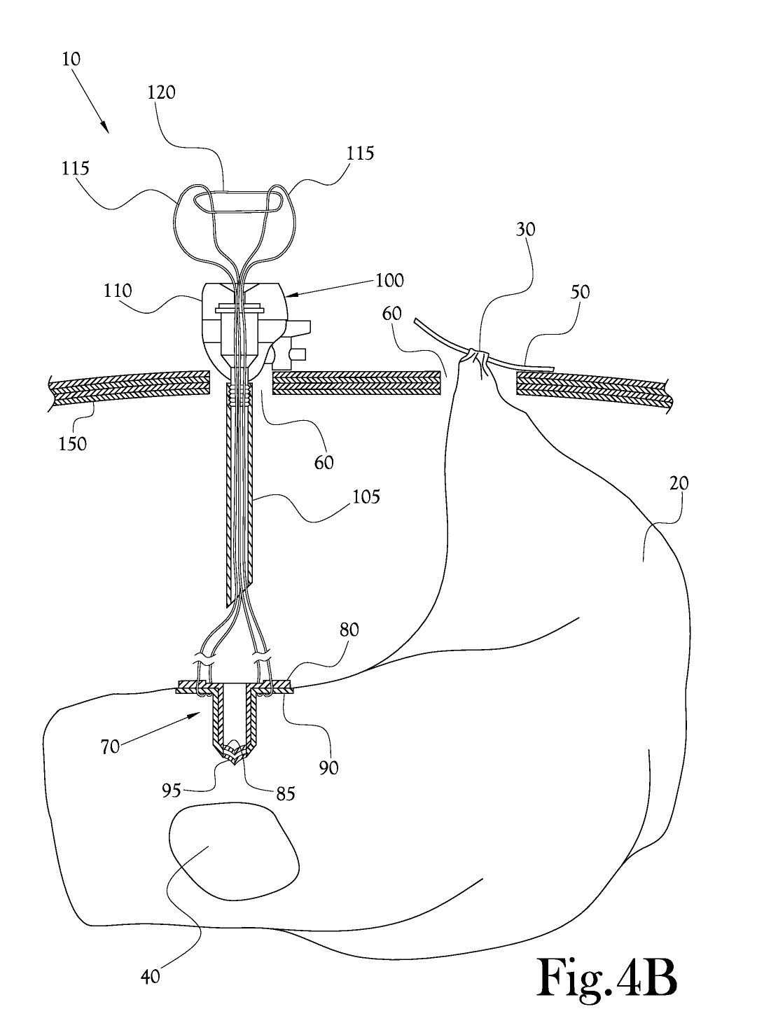

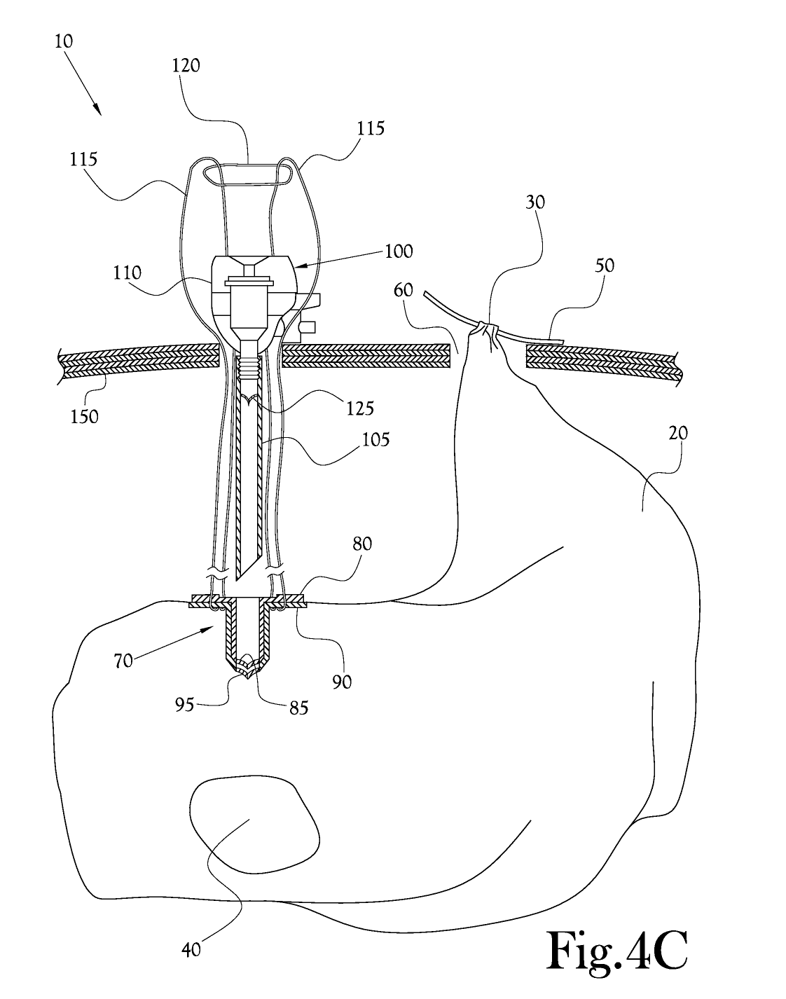

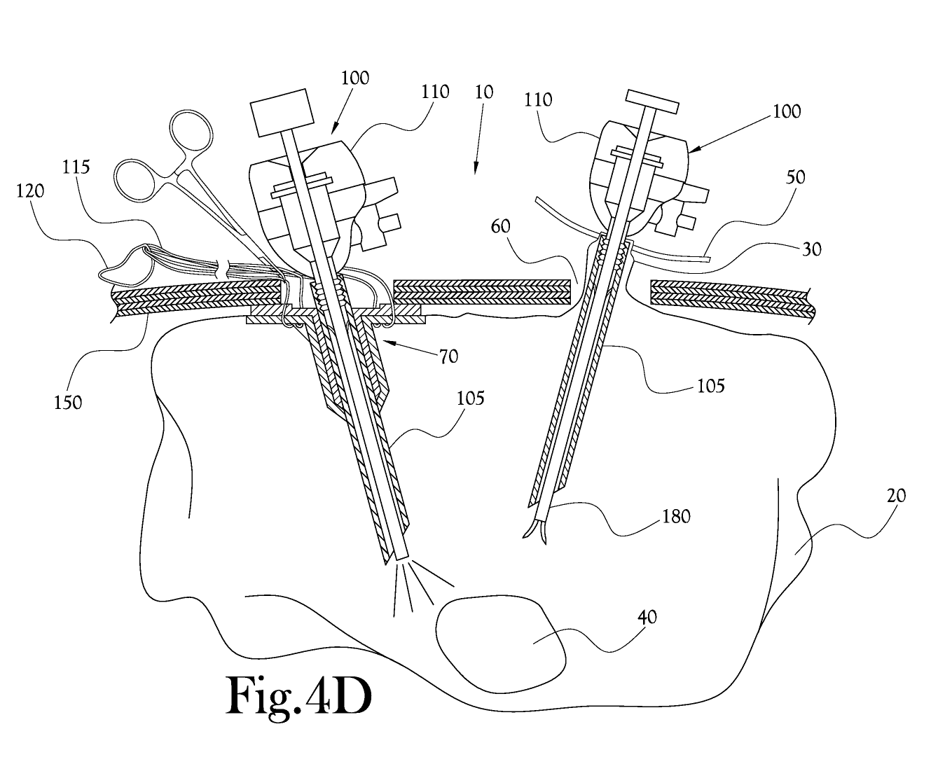

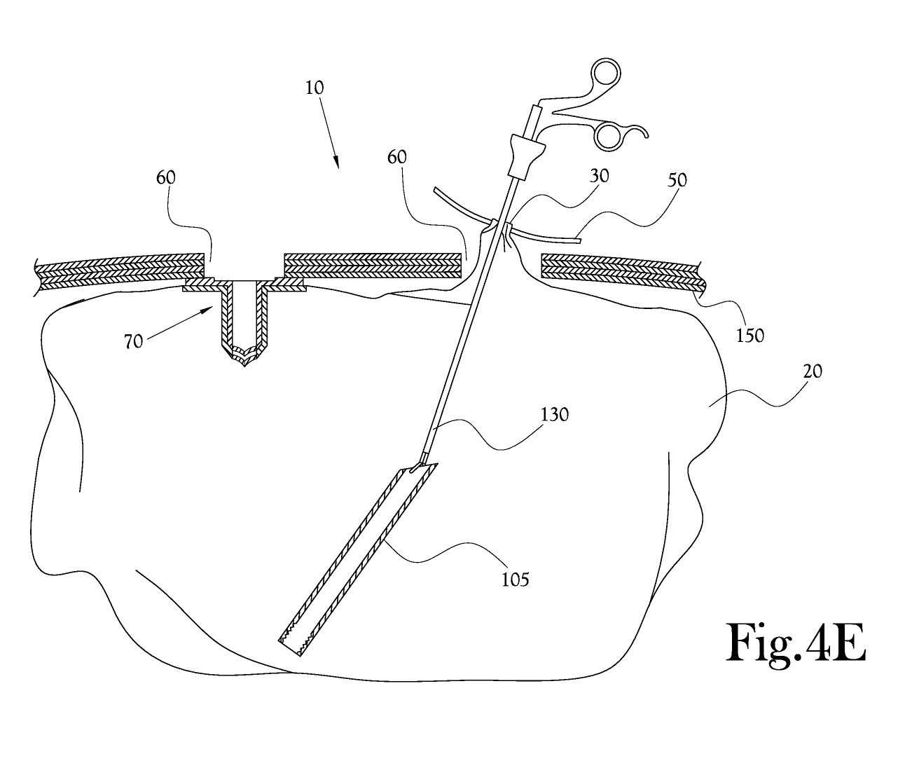

This disclosure provides systems and methods for performing morcellation of masses within a maximally expandable pelvic/abdominal morcellation containment system. The morcellation containment system 10 of the present invention comprises a morcellation containment bag 20 having a large, selectively closable, opening 30 on one lateral dimension through which a diseased organ or tumor mass 40, small or quite large, may be placed for sealed surgical morcellation. Examples of such a diseased organ or mass includes, without limitation, kidney, uterus, ovary, or other intra-abdominal/pelvic mass. Upon insertion and deployment of the morcellation containment system 10, as described herein, the diseased mass is freed from healthy surrounding tissue and is then guided into the opening 30 of the morcellation containment bag 20 with the use of the laparoscopic instruments. The opening 30 of the morcellation containment bag 20 is then sealed by way of a drawstring closure apparatus 50, which is constructed from FDA approved materials, and this drawstring-sealed opening 30 of the morcellation containment bag 20 is retrieved and brought out through any incision 60 that will accommodate the system's volume or the port chosen for morcellation. This might be the umbilicus, the cervix trans-vaginally, or a medial or lateral incision in the abdominal wall, as illustrated in FIG. 4B, for example.

The morcellation containment bag 20 is fabricated from a clear or translucent flexible and quite expandable plastic, or other FDA approved, material. The morcellation containment bag 20 includes, in an exemplary embodiment, at least three and preferably five, or more, sealed built in double squeegee type trocar access ports 70 across its anterior and lateral aspects that will each accept instruments ranging from 2-17 mm in diameter. It will be appreciated by those skilled in the art that other types of gas-isolating trocar access ports could be utilized. As seen most clearly in FIGS. 4A and 4B, the double squeegee trocar access port 70 includes a first portion 80 that overlies a portion of the exterior surface of the morcellation containment bag 20. The double squeegee access port also includes a second portion 90 that engages a portion of the interior surface of the morcellation containment bag 20. Each of the first and second portions 80 and 90 terminates in a nipple end, 85 and 95 respectively, that serves as a squeegee for the trocar 100 that passes through the port 70. The first portion 80 is adapted and has dimensions adapted to nest in the second portion 90.

Each port 70 will have at least two and preferably four suture loops secured thereto. While these loops could certainly be individual loops that would, as described below, be grasped and tugged individually, in an exemplary embodiment, four loops are defined by two looped pairs 115 of permanent suture material made of FDA approved materials. As seen in FIGS. 4A - 4E, in an exemplary embodiment, these looped pairs 115 of suture material have a secondary loop 120 to assist in gathering or grasping these suture loops with a laparoscopic instrument such as a grasper 130, as in FIG. 4A. These loops will be gathered at their apex to allow them to be grasped and brought through an abdominal laparoscopic port of a trocar 100 of any size, see FIG. 4B. Once grasped and brought through a port, the trocar 100 is removed; the loops are arranged in opposition to each other. Once the loops are in opposition to each other the prophylactic sheath, if being used, is opened externally and then the removed laparoscopic trocar 100 is reinserted through its original incision 60 traversing through the opening in the prophylactic sheath and guided towards the trocar access port 70, see FIG. 4C. Once the trocar 100 is returned to the abdomen, the laparoscope 140, or other instrument, is inserted through said trocar 100 and, with the trocar 100 proximate the trocar access port 70 and the sleeve 105 of the trocar 100 in substantial alignment with the trocar access port 70, as seen in FIG. 4C, the suture loops 115 are snugged up on top of the skin so as to bring the trocar 100 and a scope, or other instrument, through the already integrated containment system valves of the trocar access port 70 attached of the morcellation containment bag 20 and into the open operative interior volume of the morcellation containment bag 20. This procedure is completed for each trocar 100 used in the procedure. Once all trocar access ports 70 have been cannulated in this manner, the suture loops are snugged up on top of the external abdominal wall, as with a hemostat, tape, or other means so as to maintain the intra-abdominal trocar access ports 70 into direct sealed contact with the inner abdominal wall 150. This allows or causes the containment system access ports to be completely sealed to the abdominal wall 150. Internal CO2 gas expansion aids in the sealing and stability of the system.

The sealing system works as follows: 1) the trocar 100 has its own double squeegee-valved trocar access port 70 sealing the morcellation containment bag 20 against the interior of the abdominal wall 150; 2) the morcellation containment system 10 seals the trocar 100 with a double squeegee-valved trocar access port 70, or other gas-sealing access port, so as to ensure that no fluids or contamination can escape; 4) according to an exemplary embodiment trocars 100 that accompany the morcellation containment system will have sleeves 105 that can be disengaged from the upper body 110 of the trocar 100 so that the contaminated sleeve 105 can be discarded into the interior volume of the morcellation containment bag 20 without contacting exposed tissue. While use of sleeves that can be disengaged are described in conjunction with an exemplary embodiment, it will be appreciated that such sleeves are not required or mandatory, but rather may be preferred in some surgical instances. In an exemplary embodiment, the trocar sleeve 105 includes an elastomeric, one-way valve 125 for substantially preventing gases from back flowing through the sleeve when the sleeve 105 is detached from the upper body 110 of the trocar 100. In this regard, in an exemplary embodiment, the sleeves are threadably attached to the upper body 110 of the trocar 100. However, it will be appreciated by those skilled in the art that there are other means, such as a twist-lock attachment or a secure tab attachment for releasably attaching the sleeve to the upper body 110 of the trocar 100.

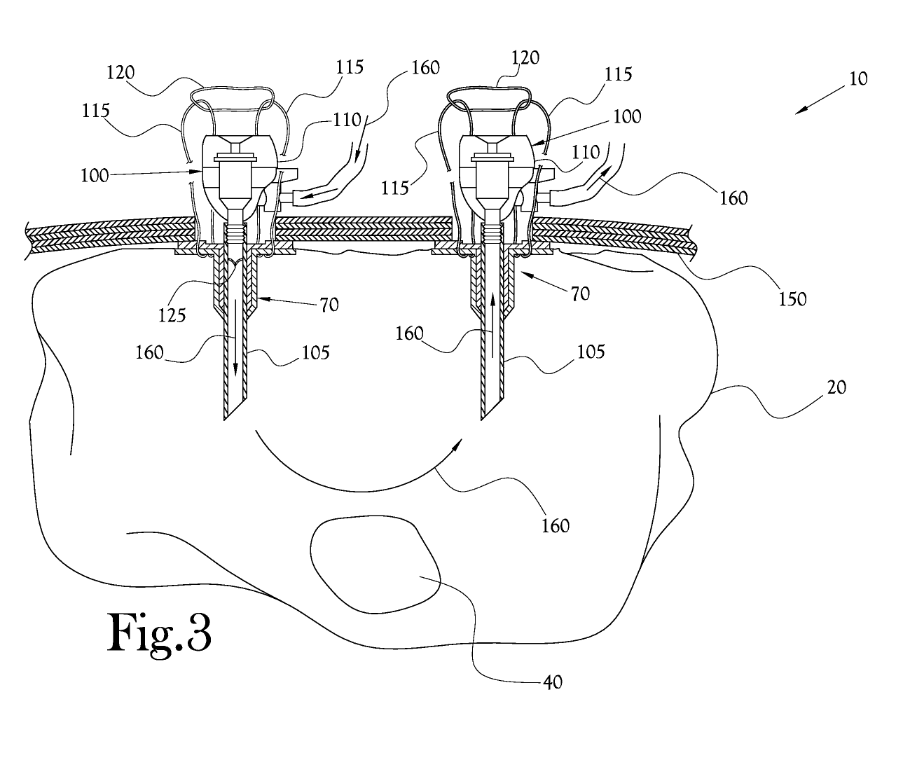

The suture loops 115 create and mandate direct contact between the trocar ports 70 and the inner abdominal wall 150. They also provide a secure locked-in-place type of immobilization for the morcellation containment bag such that the system's trocar ports 70 are completely stable during morcellation. The inner prophylactic sheath, if utilized, further protects surrounding tissue from contamination. This immobilization occurs at each port utilized during a procedure. As depicted schematically in FIG. 3, the morcellation containment bag 20 is then insufflated with a gas, preferably CO2, the standard expansion gas of laparoscopic surgery, through any laparoscopic trocar 100 that is now in fluidic communication with the interior volume of the morcellation containment bag. A steady of flow of the gas, represented by arrows 160, using an inlet port and an exhaust port, reduces the buildup of vapor within the surgical field in the interior of the morcellation containment bag of the present invention, thereby reducing the risk of vaporization hazards. Any and all ports can be utilized for this or changed at any time during a procedure or during morcellation, it being recognized that some ports and associated trocars can be utilized to introduce gas into the interior volume of the morcellation containment system while other ports can be utilized to exhaust gas from the interior volume of the morcellation containment bag.

The morcellation containment bag 20 is now expanded to fill the abdomen and pelvis under substantially the same pressure and parameters as conventional laparoscopy so as to mimic usual and standard techniques and parameters of laparoscopic abdominal wall expansion and pressure. As the morcellation containment bag 20 inflates, it acts as a mobilizer and retractor of the pelvic floor structures, the colon (large bowel), the small bowel, and the pressure applied to the surgical site by the insufflated morcellation containment bag provides a tamponade effect so as to minimize or stop bleeding or oozing at any location within the surgical site.

This morcellation containment bag 20 of the morcellation containment system 10 of the present invention is designed to completely fill the pelvis and the abdomen as allowed by the expansion of CO2 gas to the point where the containment system's surface is in direct apposition to the abdominal wall and pelvic floor further securing the systems stability during any procedure that follows. The system will only expand to fill the same volume that is allowed by usual and customary standard laparoscopic surgical techniques but at the same time retracting those mobile structures to which injury avoidance is desired. Because of the insufflation requirement of this system, continuous CO2 gas inflow will be maintained throughout any procedure that follows once the containment system 10 is insufflated. Furthermore, it will be recognized that there are state-of-the-art, commercially available electrosurgical morcellators that require continuous and immediate gas inflow and gas evacuation to minimize vaporization hazards and thereby provide for adequate visualization. In this regard, the smoke and water vapor that is created by these devices obscures visualization throughout their use during morcellation in the absence of continuous gas flow. Without the ability to have standard rapid continuous gas inflow and evacuation, the use of the electrosurgical morcellator is dangerous and not advised due to inadequate visualization.

The laparoscope, additional instruments, such as graspers, bipolar or monopolar cutters, and/or a suction-irrigator may be inserted through any morcellation containment bag trocar access port 70. Usually there will be a designated port for morcellator 180 access based upon the desires and training of the operator and a chosen skin incision (or cervical opening as desired) but any squeegee port, or other style gas-sealing port, attached to the containment system is capable of accepting the diameter of any commercially available morcellator. However, in an exemplary embodiment, the opening 30, and its associated drawstring closure apparatus 50, of the morcellation containment bag 20 is used as the access point for morcellator 180. For any location of morcellation, the drawstring aperture, if used for morcellation, provides additional skin protection by creating a circular (or conical in the event the morcellation device traverses the vagina through the cervical opening) protective platform so as to minimize contamination of the surgical field.

Once the morcellation procedure has been completed, the instruments are withdrawn from the trocars eliminating potential contamination of the field from instruments. Next, if using fully detachable trocar sleeves, the trocar sleeves 105 can then be removed from the upper body 110 of the trocar 100 and deposited into the morcellation containment bag 20 for subsequent removal via the exteriorized morcellation port, i.e. opening 30. In the absence of the valved sleeves described above, the trocar sleeves 105 are manually plugged prior to removal of the upper body 110 of the trocar 100. While several materials could be used to plug the trocar sleeve 105, the plug material should be able to resist at least 20 mm Hg of backpressure from within the morcellation containment bag. It will be appreciated that the detached sleeves 105 can either be pushed into the interior volume of the morcellation containment bag 20, or can be laparoscopically grasped and pulled into the interior volume of the morcellation containment bag 20. As the sleeves 105 are removed from the body 110 of the trocar 110 and slid into the morcellation containment bag 20, the double squeegee port system, or other suitable gas-sealing port, will keep each trocar's potential contaminants within the containment system and the port 70 will be able to contain any back-spillage. The detached sleeves 105 are then removed from the interior volume of the morcellation containment bag 20 via the exteriorized opening 30 of the morcellation containment bag 20. Once all the trocar sleeves 105 have been retrieved, if the prophylactic sheaths are being used, these can be sealed using the suture loops, the system can now be evacuated from the abdomen by releasing and securing all suture loops, releasing the gas via the drawstring closure apparatus 50, and withdrawing the morcellation containment bag 20 through its insertion port. In the event trocars with non-detachable sleeves are being used, the suture loop prophylactic sleeve can be sealed and withdrawn into the containment bag.

While a trocar 100 having a detachable sleeve 105 has been described in conjunction with an exemplary embodiment, it will be appreciated that in an alternate embodiment, a prophylactic sheath (not shown) could be utilized. The sheath would be pulled up through the belly wall prior to inserting the trocars into the morcellation containment bag. At the conclusion of the procedure, completion of the morcellation procedure, the trocar would be removed from this prophylactic sheath, leaving the prophylactic sheath in place. The suture loops would be utilized to pull the upper portion of the sheath back through the sleeve, folding the sleeve inward upon itself into the interior volume of the morcellation containment bag.

Thus, it will be appreciated that a morcellation containment system has been described which provides numerous advantages over known morcellation systems. In this regard, the morcellation containment system of the present invention provides a morcellation containment system which is sealed to the inner surface of the abdominal wall and which is inflatable so as to retract or mobilize vital organs from the surgical field. Additionally, the morcellation containment system of the present invention can be accessed by a standard laparoscope thereby providing direct visualization of the morcellation process. In accordance with the present invention, continuous gas flow within the interior volume of the morcellation containment bag allows the use of any type of morcellator, including without limitation, an electrosurgical morcellator. Further the morcellation containment system of the present invention provides a morcellation containment system which specifically addresses the needs of and allows for trans-cervical, as well as trans-vaginal morcellation.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A morcellation containment system for isolating and containing a diseased tissue mass for morcellation, said morcellation containment system comprising:
a morcellation containment bag defined by an expandable body adapted to form a substantially sealed enclosure to allow a surgical procedure to be performed within;
a plurality of gas-sealing ports adapted to allow passage of a surgical instrument into the cavity of said morcellation containment bag;
means for manipulating each said port and drawing said port into close proximity with an interior lining of an abdominal wall, thereby securing said bag in a selected position.

2. The morcellation containment system of claim 1, wherein said bag is adapted to be insufflated with a gas so as to expand said bag thereby allowing an exterior portion of said bag to contact an inner abdominal wall.

3. The morcellation containment system of claim 1 wherein said means for manipulating each said port and drawing said port into close proximity with an interior lining of an abdominal wall is defined by at least one suture loop.

4. The morcellation containment system of claim 3, wherein said port is sealed against the inner abdominal wall of a patient with said tether.

5. The morcellation containment system of claim 1, wherein each of said ports includes a first portion that overlies a portion of an exterior surface of said morcellation containment bag and a second portion that engages a portion of an interior surface of said morcellation containment bag, wherein each of the first and second portions terminates in a nipple end thereby defining a double squeegee.

6. The morcellation containment system of claim 1, wherein said bag is substantially transparent.

7. The morcellation containment system of claim 1 wherein said morcellation containment system further comprises a trocar having an upper body and a selectively detachable lower sleeve.

8. A morcellation containment system for isolating and containing a diseased tissue mass for morcellation, said morcellation containment system comprising:
a morcellation containment bag defined by an expandable body adapted to form a substantially sealed enclosure to allow a surgical procedure to be performed within;
a plurality of gas-sealing trocar access ports adapted to allow passage of a surgical instrument into the cavity of said morcellation containment bag;
at least two suture loop members coupled with each of said plurality of ports for manipulating each said port and drawing said port into close proximity with an interior lining of an abdominal wall, thereby securing said bag in a selected position.

9. The morcellation containment system of claim 8, wherein said bag is adapted to be insufflated with a gas so as to expand said bag thereby allowing an exterior portion of said bag to contact an inner abdominal wall.

10. The morcellation containment system of claim 8, wherein said trocar access port is sealed against the inner abdominal wall of a patient with said suture loop.

11. The morcellation containment system of claim 8, wherein said bag is substantially transparent.

12. The morcellation containment system of claim 8 wherein said morcellation containment system further comprises a trocar having an upper body and a selectively detachable lower sleeve.

13. The morcellation containment system of claim 8 wherein said trocar access port includes a first portion that overlies a portion of an exterior surface of said morcellation containment bag and a second portion that engages a portion of an interior surface of said morcellation containment bag, wherein each of the first and second portions terminates in a nipple end thereby defining a double squeegee for the said surgical instrument.

14. The morcellation containment system of claim 13 wherein said first portion of said trocar access port is adapted to nest within said second portion of said trocar access port.

15. A morcellation containment system for isolating and containing a diseased tissue mass for morcellation, said morcellation containment system comprising:
a morcellation containment bag defined by an expandable body adapted to form a substantially sealed enclosure to allow a surgical procedure to be performed within;
a plurality of gas-sealing trocar access ports adapted to allow passage of a surgical instrument into the cavity of said morcellation containment bag, wherein each of said trocar access port includes a first portion that overlies a portion of an exterior surface of said morcellation containment bag and a second portion that engages a portion of an interior surface of said morcellation containment bag, wherein each of the first and second portions terminates in a nipple end thereby defining a double squeegee for the said surgical instrument;
at least two suture loop members coupled with each of said plurality of ports for manipulating each said port and drawing said port into close proximity with an interior lining of an abdominal wall, thereby securing said bag in a selected position.

16. The morcellation containment system of claim 15, wherein said bag is adapted to be insufflated with a gas so as to expand said bag thereby allowing an exterior portion of said bag to contact an inner abdominal wall.

17. The morcellation containment system of claim 15, wherein said trocar access port is sealed against the inner abdominal wall of a patient with said tether.

18. The morcellation containment system of claim 15, wherein said bag is substantially transparent.

19. The morcellation containment system of claim 15 wherein said morcellation containment system further comprises a trocar having an upper body and a selectively detachable lower sleeve.

20. The morcellation containment system of claim 15 wherein said first portion of said trocar access port is adapted to nest within said second portion of said trocar access port.
